(19) United States
(12) Patent Application Publication
(10) Pub. No.: US 2007/0280652 A1
(43) Pub. Date: Dec. 6, 2007

(54) Tobacco vaporizer and related water pipe system

(76) Inventor: Clayton J. Williams, San Marcos, TX (US)

Correspondence Address: JOHN K. BUCHE, 7777 FAY AVENUE, SUITE 205, LA JOLLA, CA, 92037, US

(21) Appl. No.: 11/443,386

(22) Filed: May 31, 2006

(51) Int. Cl. A61L 9/03; A61H 33/06

(52) U.S. Cl. 392/386; 392/394

(57) ABSTRACT

An improved vaporizer unit and water pipe assembly for extracting ingestible vapors from tobacco and herbs. Preferable embodiments of said assembly may comprise blown glass, colored glass, and adaptations for the safety of the user and to protect against breakability of said assembly. Other preferred features and embodiments include but are not limited to a cage secured to protect a central vessel housing a heating means, a hanger for storing, enhanced airflow, enhanced draw, and portability.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of Invention

The present application relates to the fields of water pipes and tobacco vaporizers.

2. Background of the Invention

The general concept of a water pipe has been in existence for centuries. Water pipes are also sometimes referred to as “Narghiles” or “hookahs,” with original popularity of the devices tracing back to Turkey around the time of Murat the IV around 1623-40, where they became an important part of coffee shop culture and a preferable means of smoking tobacco. (http://www.thehookah.com/history/1.htm). Typical hookah tobaccos would include a mixture of shredded tobacco leaf mixed in with a sweetener such as honey, molasses or semi-dried fruit, although more modern tobaccos feature glycerin as a primary sweetener because of humectant properties and the ability to readily mix with tobacco flavors, such as apple, strawberry, vanilla, mint, etc. (http://en.wikipedia.org/wiki/Hookah). Water pipes are now also commonly used to extract active medicinal vapors from legal herbs such as eucalyptus, hops, chamomile, lavender, lemon balm, sage, thyme and many more.

The function of a traditional water pipe is that when a smoker inhales through a tube, a pressure difference forces air past a lit coal, igniting tobacco that gives off smoke, which smoke is then pulled away from the tobacco, through a chamber of water and into the air of a smoke chamber-whence it is inhaled by the smoker. The smoke is delivered, substantially filtered of tar and nicotine, and of a pleasurable temperature cool to the user. The basic components of a water pipe or traditional Narghile have generally included a base or smoke chamber, which is partially filled with water; a bowl, which contains the tobacco; the heating apparatus, which is typically placed on top; the pipe, which connects the bowl to the base by a tube that plunges into the water; and a hose, which connects to a second tube in the pipe that does not plunge into the water, but only the air of the smoke chamber. A variety of adaptations have been made to this most basic arrangement.

In recent years, it has been discovered that use of heating means, other than fire, or coals may be beneficial in the production of thicker vapor clouds, that are more flavorful, and with minimal toxic contaminants. These other devices are frequently referred to as “vaporizers.” Cigarettes, for instance, rely on a traditional incendiary device to light and continually smolder tobacco to extract flavor and active substances, such as nicotine. Smoke from cigarettes is loaded with products of combustion, many of which are known carcinogens. By contrast, vaporizers extract active compounds and flavors from tobacco products in a manner that typically results in fewer carcinogens and known health hazards such as tar, nitrosamines or soot-containing solids. Vaporizers operate principally by maintaining temperature just above the point of vaporization, but below the temperature point of combustion, so that important active plant substances are extracted directly to vapor and so that other combustible products are left behind.

One popular vaporizer on the market is the “Volcano” vaporizer manufactured by Stortz & Bickel of America, Inc. (http://www.vapormed. de/us_home.htm?x=93&y=4), which is described in U.S. Pat. No. 6,513,524 to Stortz (2003) and in a series of related German patents. This “Volcano” vaporizes active plant substances by pumping air through a heated aluminum block, similar to an oven with aeration ducts, and by then delivering vapors to a balloon through which the user inhales the vapors. Water is not employed in the process, so the apparatus does not operate in the manner of a Narghile or “hookah” water pipe. Drawbacks of this product include, but are not limited to the absence of water filtration, so vapor is neither cooled, nor filtered of dust and metal oxides from a heating element.

Another available vaporizer product is the VaporBrothers® vaporizer, viewable at http://www.vaporbrothers.com/pages/homepage.html. This is a vaporizing product, but does not employ any significant form of water filtration technology. Limitations of this product also include, but are not limited to restricted draw, narrow airflow, and the absence of water filtration so vapor is neither cooled, nor filtered of dust and metal oxides from a heating element.

As described on http://www.vaposcience.com/and on http://www.aromed.com/en/aromed.html, another commercially available vaporizer product is the “AroMed” Vaporizer, which does employ water filtration. This vaporizer uses a light bulb to generate a hot air source. However, while the AroMed product has some use for herbal therapy, it performs in a different manner, and suffers from structural limitations that effect performance. The Aromed vaporizer does not adequately protect the user from heat or the device from breakage. Further, airflow in this product is restricted because of inadequate ventilation and narrow components that require a user to use excess suction to draw vapors through the components. The product has restricted vapor yield, is primarily stationary and must sit on a table. Other limitations are that this device exposes the user to bright light and heat from the bulb, thus presenting both a safety and aesthetic limitation.

### SUMMARY OF THE INVENTION

Accordingly, it is an objective of the present application to provide an improved vaporizer and related water pipe system.

It is yet another object of the present application to provide a tobacco and herb vaporizer with improved airflow and for producing thick vapor clouds. Embodiments disclosed in the present application employ passageway components and air flow apertures with sufficient diameter to allow a fast and smooth draw.

It is a further object of the present application to provide a water pipe featuring a halogen bulb element that produces clean, metal-oxide free heat.

It is another preferable object of the present application to provide a water pipe and vaporizer assembly that are uniquely designed to be user friendly (avoiding burns to the user), which is easy to clean, and stores easily. A preferred embodiment of this application further features an assembly that is portable and facilitates passing to other users.

It is a further object of the present application to provide for a light gun apparatus that causes beneficial room illumination, the appearance of color changes on glass surfaces and components of the water pipe.

It is a further object of the present invention to provide large yields of vapor from relatively small quantities of herbs and tobacco products.

It is a further object of the present invention to provide a superior means of extracting active vapors from herbs such as eucalyptus, hops, chamomile, lavender, lemon balm sage, thyme, and many more.

It is a further object of the present invention to provide a temperature control through use of dimmer switches.

It is a further object of the present invention to provide a stylized water pipe with some hand blown components to be used in conjunction with a light activated vaporizer. An embodiment also preferably features thick colored glass, which is aesthetically pleasing to the user.

Other objectives of the invention will become apparent to those skilled in the art once the invention has been shown and described. These objectives are not to be construed as limitations of applicant's invention, but are merely aimed to suggest some of the many benefits that may be realized by the apparatus of the present application and with its many embodiments.

### BRIEF DESCRIPTION OF THE DRAWING

The manner in which these objectives and other desirable characteristics can be obtained is explained in the following description and attached drawings in which:

FIG. 1 is a front perspective drawing of a preferable vaporizer pipe assembly.

FIG. 2 is an isometric view of the vaporizer unit of the present invention cradled on a safety and storage hanger assembly while disconnected from the remainder of the water pipe assembly.

FIG. 3 is a perspective view of the vaporizer unit of the present invention standing alone and featuring a preferable cage embodiment.

FIGS. 4A and 4B comprise an exploded side view of the vaporizer unit of the present invention (4A), further depicting how the vaporizer unit (4A) is preferably insertable into a bowl unit (4B).

FIG. 5 is a side perspective view of the vaporizer unit, demonstrating how a preferable handle is affixed thereto and permits threading of the cord(s) from the power source to a heating means. FIG. 5 further depicts a preferable ventilation feature and vent covering on the vaporizer unit.

It is to be noted, however, that the appended drawings illustrate only typical embodiments of this invention and are therefore not to be considered limiting of its scope, for the invention may admit to other equally effective embodiments that will be appreciated by those reasonably skilled in the relevant arts. Also, drawings are not necessarily made to scale but are representative.

### DETAILED DESCRIPTION OF THE INVENTION

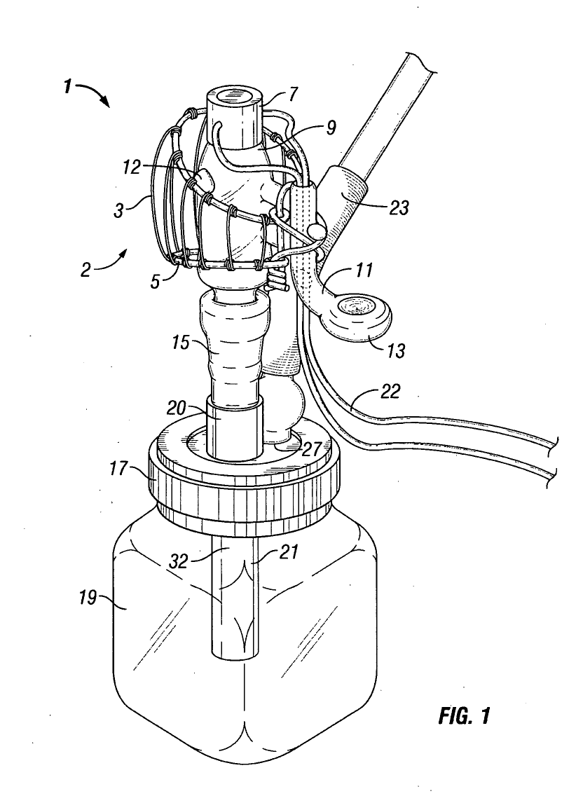

FIG. 1 is a perspective view of a preferable vaporizer pipe assembly 1. Basic components of the shown assembly comprise a vaporizer unit 2 with a heating means 7, where the vaporizer unit 2 is removably insertable into a bowl unit 15. The bowl unit 15 is a reservoir for tobacco, herbs, or other items from which vapor is extracted. The bowl unit 15 inserts into a stem 32, which stem is formed from an upper stem 20 and lower stem 21. The stem 32 extends into a chamber 19 with upper stem 20 and lower stem 21 separated by a preferable chamber top 17. The stem 32 will also be typically secured through chamber top 17 by use of a latex, rubber, cork, or synthetic rubber means, including grommets. Chamber top 17 typically features a plurality of holes formed therein, which serve to accommodate insertion by the stem 32 connected to the vapor source, chamber outlet 27, and an optional “carb” or “choke” hole. It should, however, be noted that a “carb” is not necessary with the disclosed assembly 1 because of the operation of the vaporizer and because a user can simply remove central vessel 9 from bowl unit 15 and clear vapor from the chamber. Applicant has found preferable distances of 10 to 30 millimeters between respective holes to be preferable, and also that holes may be angled toward one another. Vapor from operation of the vaporizer pipe assembly is drawn by sucking action of the user down through the bowl unit 15, through the stem 32 and into a chamber 19, which chamber 19 typically contains liquid through which vapors are filtered. The distal portion of stem 32 is typically submersed beneath the liquid in the chamber 19. Chamber liquids may vary, but are typically water, mineral water, juices, alcohol, or any variety of flavor containing beverages. The chamber 19 may also preferably contain ice, which tends to cool incoming vapors. Vapors enter the chamber 19, which vapors may have been filtered through liquid, then proceed to and through chamber outlet 27 where they are drawn by the user through a preferable hosepipe 23, the end of which may preferably employ a mouthpiece. Hosepipe 23 may be formed in multiple segments, which in total will typically be in the range of 50 to 200 millimeters in length being preferable to achieve beneficial airflow, and wherein the inside diameter of a hosepipe 23 is preferably in the range of 7 to 15 millimeters at a narrowest point along the hosepipe, with a still further preferable diameter being 10 millimeters. Applicant has found that the vaporizer pipe assembly 1 preferably will also operate with superior draw when the inside diameter of all components is equal to or greater than 10 millimeters. Bulbous segments may be added to the hosepipe 23 to increase airflow, or to modify the pipe's draw. The hosepipe 23 may additionally feature flexible sections for user convenience. The hosepipe 23 may additionally employ a preferable backflow prevention valve, such as manufactured by Bel-Art Products, Inc., with airflow check valves with a tubing taper of 9/16 to ⅜ inches. This backflow valve may be secured in a mouthpiece, hosepipe 23, or at a position disposed in the chamber top 17.

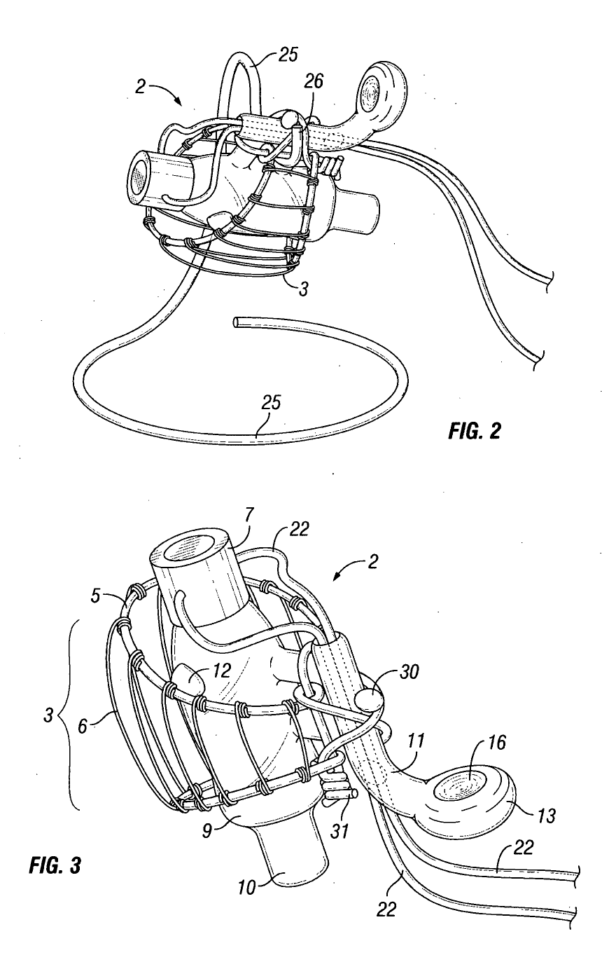

Another feature viewable in the embodiment of FIG. 1 is a protective cage 3 surrounding the vaporizer unit 2, which cage helps to protect the user from burns, which may also protect the vaporizer unit 2 from damage or breakage, and which may also facilitate storage of the vaporizer unit 2 on a hanger 25 (FIG. 2) when not assembled or in use. A handle 11 is featured affixed to vaporizer unit 2, with the handle featuring a grip 13, and wherein the handle further features a preferably hollow passageway through which cord(s) 22 that are connected to a power source 29 may pass through to supply power to heating means 7.

FIG. 2 is an isometric view of the vaporizer unit 2 as it appears when preferably hung on a hanger 25 when not in immediate use. Vaporizer unit 2 is typically formed from glass, so it can be subject to breakage. Vaporizer unit 2 is also hot to the touch after use. Therefore, a hanger 25 is a very beneficial embodiment for use with the vaporizer unit 25 to avoid bums, to avoid breakage of the product, or for simply storing the vaporizer unit 25 when the remaining portions of the vaporizer pipe assembly 1 are being used or cleaned. The hanger 25 embodiment shown in FIG. 2 defines a snaking shape, where the lower portion forms a partial circle, or coil for support, and where an upper portion of the hanger 25 rises steeply from the coil to form a hook 26 to which the vaporizer unit 2 may be attached, preferably by hanging. The hook 26 may secured to the handle 11 or to any portion of cage 3. A preferable hook 26 would be in the range of 20 to 50 millimeters of hanger 25. For ideal support of vaporizer unit 2, hanger 25 has a base width of a range of 100 to 200 millimeters in width and a height of up to 200 millimeters. Those skilled in the arts will appreciate that a hanger 25 may assume a variety of forms, however, with a primary function being to suspend for convenience and safety the vaporizer unit 2. The hanger 25 is also a good place to suspend the vaporizer unit 2 as it is warmed up for use. The hanger 25 may be formed of any of a variety of materials that may include, but are not limited to plastic, metal, metal alloys, powder-coated or covered metals, plastics or alloys. Applicant has found copper wire particularly suitable for use in forming a hanger 25.

FIG. 3 is a side perspective view of the vaporizer unit 2, showing in more detail a preferable embodiment featuring cage 3. As indicated in previous paragraphs, cage 3 is beneficial inasmuch as it shields the user from burns, serves to protect the vaporizer unit 2 from breakage, and because it may also serve as a location for hanging of the vaporizer unit 2 from a hanger 25. Other benefits will be appreciated by those skilled in the arts. With these listed benefits in mind, a cage 3 may take many forms, but the embodiment depicted in FIG. 3 features a preferable shape defined by predominantly circular or basket shape that envelops the vaporizer unit 2. This embodiment features a cage 3 with a frame member 5, further comprising upper and lower sections with a plurality of cross members 6 affixed therebetween. Cross members 6 serve for support, decoration, and for safety of both the user and vaporizer unit 2. Said cross members 6 may also take the form of one woven piece of wire to make many cross members. Cage 3 is preferably formed from any of the groups of materials, including but not limited to, metals, alloys, copper, aluminum, plastics, combinations thereof, or composite materials, with or without protective coatings. A cage 3 might also readily take the form of a guard of uniform construction, for instance, Plexiglas® plastic, or other plastic shield, including but not limited to acrylic, PVC, polycarbonate, vinyl, polyethylene, polypropylene or other plastic. The cage 3 of the present figure is fixed securely or removably to vaporizer unit 2, and preferably at or near handle 11. The cage 3 may also be fixed securely, or removably (if desired) to the vaporizer unit 2 by a fastening means 31. The fastening means 31 shown in FIG. 3 is a metal wire twisted around and through handle 11 and cage 3 and securing upper and lower segments of frame member 5 to one another. In yet another embodiment of cage 3, there is a frame member 5 with upper and lower sections (preferably of “C” shapes) fixedly secured to one another by both a U-shaped backbone and a front arm piece, both of which extend between and connect the upper and lower sections of frame member 5. The front arm piece ranges from 50 to 80 millimeters in height and the U-shaped backbone ranges from 15 to 40 millimeters in height, the latter of which U-shaped backbone is typically cut from wire in the range of 80 to 150 millimeters in overall length. In this embodiment, upper section and lower sections of frame member 5 are bent into “C” shapes from wires preferably 200 to 280 millimeters in length. Applicant has found that upper and lower sections, U-shaped backbone, and front arm piece are preferably formed from ten-gauge copper wire, and that twenty-gauge or smaller solid copper wire may be wrapped around and between upper and lower sections approximately 10 to 30 millimeters apart to keep fingers from contacting the hot surface of vaporizer unit 2. Other gauges of wire might also be suitable. A fastening means 31 wraps around and securely fixes U-shaped backbone of cage 3 to vaporizer unit 2, preferably around handle 11. Also shown is a knob 30, which is a preferable useful feature, typically formed of glass (but other materials may be used) to aid with attachment of the cage 3 or fastening means 31 to vaporizer unit 2.

FIG. 3 further depicts a handle 11 featuring a hollow channel extending therethrough for power cords 22. The handle 11 features holes, typically in the range of 3 to 10 millimeters in diameter through which the cord(s) 22 may pass to the heating means 7. The handle 11, as well as the vaporizer unit 2 are preferably formed from blown glass, so these cord holes may be formed when the glass is blown. A grip 13 is preferably affixed to handle 11, with an optional indention 16 particularly well suited for a user's thumb. A preferable handle 11 length is in the range of 40 to 80 millimeters in height and may also be spaced from the central vessel 9 in the range of 12 to 40 millimeters by use of at least one side support(s) 14. This distance from the central vessel 9 has been found to be adequate to protect the user's fingers and facilitate ease of use. At least one side support(s) 14 are also employed to fixedly connect handle 11 to central vessel 9, with preferable diameters of such side support(s) typically ranging from 8 to 12 millimeters. Where an “L” shaped handle 11 is employed, the lower portion of the “L” is of an outwardly extending length in the range of 20 to 50 millimeters.

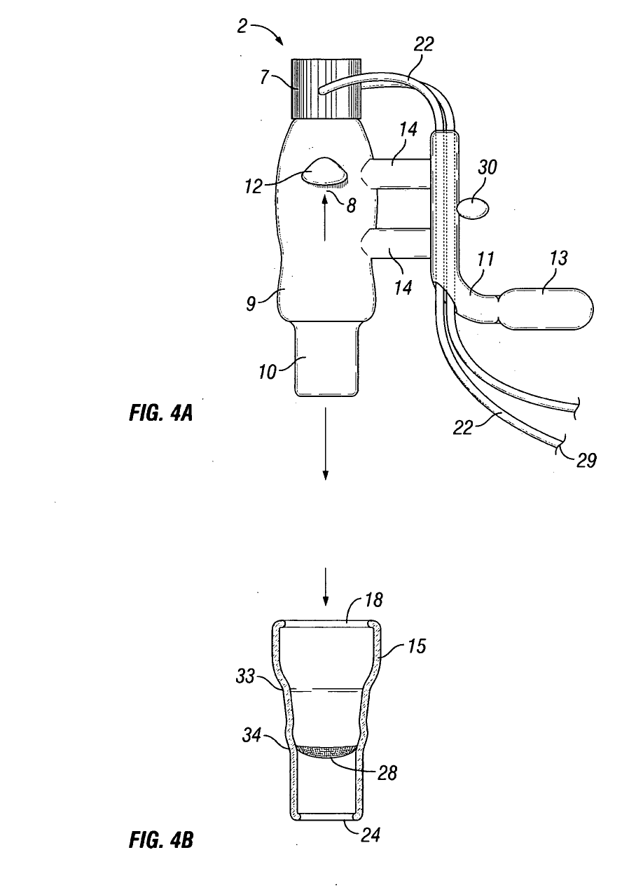

FIGS. 4A and 4B demonstrate a typical function of an embodiment of the application and how vaporizer unit 2 is insertable into bowl unit 15. This embodiment of vaporizer unit 2 is formed from a central vessel 9, which is preferably glass blown, and further defining a lower vessel 10, which lower vessel is of a shape removably insertable into an upper opening 18 of bowl unit 15. FIG. 4A shows how lower vessel 10 is typically of a lesser circumference than central vessel 9. Also, FIG. 4A shows side supports 14, which serve to fixedly connect handle 11 to the body of central vessel 9, and which side supports may also be formed of blown glass. FIG. 4A also shows an “L” shaped handle 11, which Applicant has found to be a useful shape. A suitable attachment point for a knob 30 is at a central point along handle 11 to best accommodate balanced hanging of vaporizer unit 2 on hanger 25 (FIG. 2).

The vaporizer unit 2 shown in 4A largely serves the purposes of raising the temperature within the central vessel 9 to a point where active ingredients in plant substances, herbs and tobacco will be turned to vapor, which temperature is in a preferable range of 266 to 374 degrees Fahrenheit (130-190 degrees Celsius). To generate the heat required to form vapor, a heating means 7 is used, which is connected by power cord(s) 22 to a power source 29, which power source 29 is typically a wall outlet, although other battery sources are contemplated. The heating means 7 also works with a bulb 4, preferably halogen, and which heating means 7 may also employ a dimmer switch to adjust the intensity of the vaporizing heat to accomplish maximal vaporization and at a level preferable to the user. Preferable bulbs are 100 to 200 watt bulbs. One preferable bulb 4 is a 100-watt halogen bulb, with mini-candela screw base, such as the Light Bulbs Etc., Inc. (Item Number JD-Q100T3/CL/MC/130V). A preferable dimmer is of the variety such as the Credenza Lamp Dimmer by Lutron, Model No.TT-300 for incandescent lamps. A hole in the top of vaporizer unit 2 is preferably in the range of 10-20 millimeters, which best accommodates the bulb 4 and heating means 7. To generate adequate vapors within the vaporizer unit 2 and to have the correct draw on the vaporizer pipe assembly 1, adequate supply of air is also required, which is accomplished by and through at least one air flow aperture(s) 8 formed through a wall of central vessel 9. Air flow aperture(s) 8 are preferably in the range of 2 to 10 millimeters in diameter, with a further preference being apertures of 2-3 millimeters. A vent covering 12 is featured on this embodiment, with this vent covering 12 being of a preferable width range of 5 to 25 millimeters and occurring at a preferable distance from the top hole in the central vessel 9 in a range of 20 to 30 millimeters. Corresponding air flow aperture(s) 8 are also preferably located a distance down from the top hole in the central vessel 9 in a range of 20 to 30 millimeters. They may also be located on opposite sides of central vessel 9. Vent coverings 12 are preferably made of blown glass. Air flow apertures 8 are typically blown out first, then pushed in with a glass blowing tool, thus creating a depression with a hole in it, and whereupon a vent covering 12 may then be fashioned. The bulb 4 of the heating means 7 also is situated far enough above the bowl unit 15 to maximize air flow around the bulb 4 and generate thick vapor clouds. A typical halogen bulb used by this embodiment would be in a range of 35 to 60 millimeters in length, so the central vessel 9 should accommodate the bulb with adequate space to circulate air and extracted vapors. The central vessel 9 of the vaporizer unit 2 is of a height in a range of 50 to 100 millimeters and featuring a maximum central width in a range of 25 to 60 millimeters in width. The lower portion of the vaporizer unit 2, which is labeled as lower vessel 10 is preferably of height in the range of 15 to 35 millimeters and featuring a hole at its base so that base is in the range of 10 to 30 millimeters in total diameter. The width of this hole at the base of lower vessel 10 is such that it will readily insert into upper opening 18 of bowl unit 15.

FIG. 4B is a cross sectional view of bowl unit 15, which features an upper opening 18, a lower opening 24, and screen 28 situated therebetween. The substance (tobacco, herbs) to be vaporized is placed on the screen 28 by the user, the vaporizer unit 2 is then inserted into the bowl unit 15, and the heating means 7 is activated (if it has not already been activated previously), thereby, generating vapors from the substances heated. A user may typically warm up the vaporizer unit 2 for 10 to 20 minutes prior to using to accomplish vaporization. This vapor may then be drawn down through the vaporizer unit 2 by suction of the user, through the bowl unit 15, stem 32, chamber 19, through chamber outlet 27, and then hosepipe 23 after which point the user may consume the vapors. Bowl unit 15 has its upper opening 18 being of a diameter typically in the range of 20 to 30 millimeters, with up to 25 millimeters in width being a further preferable diameter. A total diameter of the top of bowl unit 15 is in a range of 30 to 40 millimeters. The inside surface of bowl unit 15 defines a lower vessel shelf 33, suited for lower vessel 10 to rest on when the vaporizer unit 2 is inserted therein. This lower vessel shelf 33 occurs at a location preferably in the range of 20 to 30 millimeters down from upper opening 18, with a further preferable distance being 25 millimeters down from upper opening 18. This lower vessel shelf 33 occurs suitably at a point inside the bowl unit 15 where the interior diameter measures approximately 18 millimeters, although a range of 15 to 20 millimeters would also be typical. At a location within bowl unit 15 in the range of 10 to 30 millimeters down, when measured from lower vessel shelf 33, there is preferably located a screen receiving means 34 (preferably a screen shelf). Yet another preferable distance for the screen receiving means 34 to occur is 15 millimeters when measured down from lower vessel shelf 33. Yet another way to state this suitable dimension is that the base of the lower vessel 10 (which is part of vaporizer unit 2 containing the bulb) is in a range of 10 to 30 millimeters from the screen. Further, the tip of the bulb 4 is preferably in the range of 50 to 85 millimeters from the screen 28. This is a preferable distance between the heat source and screen to vaporize plant materials and enjoy suitable vapor production. The diameter of lower opening 24 and the interior diameter space of the bowl unit 15 beneath screen 28 is typically in the range of 12 to 14 millimeters. The lower portion of bowl unit 15 inserts into a stem 32 that extends into chamber 19, which chamber is preferably a clear glass vessel with a total volume in the range of 0.3 to 2 liters, with 0.85 liters being further preferable. A typical height for the bowl unit 15 is in the range of 70 to 130 millimeters. Stem 32 may be formed of a variety of materials, including but not limited to glass, plastics, metal, rubber or rubber synthetics, however applicant has found a latex hose preferable for the upper stem 20, with a 50-65 millimeter length, and approximate 34 inch inside diameter. Lower stem 21 may also be formed from any of the materials of suitable equivalents, but applicant has found 19-millimeter borosilicate clear tubing to be preferable.

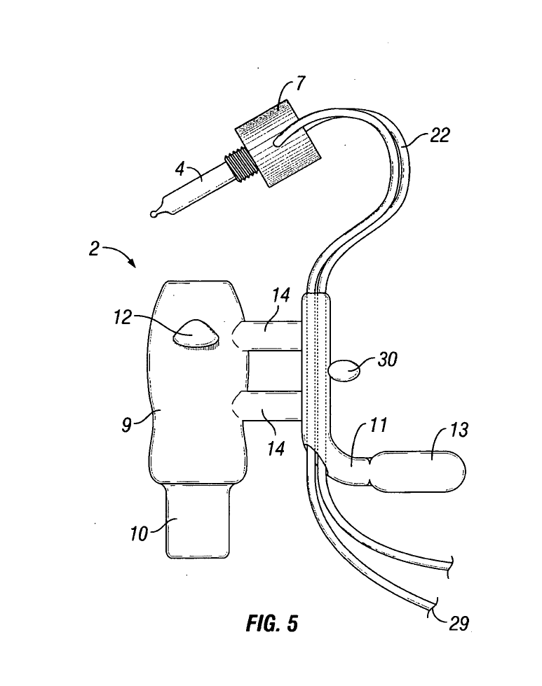

FIG. 5 is a side view of the embodiment of FIG. 4A, but showing the heating means 7 when not inserted into the top portion of vaporizer unit 2. The preferred heating means for vaporizing substances is bulb 4, which is also typically a halogen bulb. During use, heating means 7 is fixedly secured to the top portion or vaporizer unit 2. The heating means 7 may be secured by a variety of means, including but not limited to snapping into place, clamps, or by screwing in by a threadable means. Applicant has found a preferable securing to be accomplished by a snug fit of heating means 7 to the hole defining a top portion of vaporizer unit 2.

Many of the principal components of the vaporizer pipe assembly 1 are preferably featured in blown glass. These parts may include but are not limited to portions of the vaporizer unit 2, namely, the vaporizer handle 11, central vessel 9, lower vessel 10, side supports 14, vent covering 12, air flow aperture 8, bowl unit 15, stem 32, chamber 19, or hosepipe 23. It should be noted that pipe assembly 1 may also feature components that are not hand blown, but rather could be commercially available molded glass components, for instance chamber 19. Applicant has further found that the use of colored glass contributes to a pleasing visual sensation while using the vaporizer pipe assembly. For instance, when red glass is used in the components to the vaporizer unit 2, the bulb 4 of the heating means 7 generates a deep red glow throughout the room where the vaporizer pipe assembly 1 is in use. Other colors may be contemplated, or clear components as well. It should be noted that in a preferable embodiment featuring colored glass, the central vessel 9 will typically change colors when heated because of interactions with color treatments of the glass, light and heat. For instance, the central vessel 9 can change colors as heated or cooled from yellow to orange, orange to red, red to dark red/brown and vice versa. A typical color change could be expected as a pipe cools over approximately five minutes. Applicant has found that certain colors are preferable to accomplish the color changes described and also to form a suitably opaque color to adequately shield the user from the bright light and heat from a halogen bulb, such colors including but not limited to cadmium metal containing colors marketed by Glass Alchemy, Ltd. (www.glassalchemyarts.com), such as red crayon #1104, orange crayon #1204, and yellow crayon #1304, olive green crayon #1406, chartreuse crayon #1403, chocolate crayon #1804. Applicant has further found that for colors to serve their intended purposes, at least two layers, and preferably three layers should be applied prior to blowing out the shape of central vessel 9.

Disclosed is a vaporizer pipe assembly comprising a vaporizer unit featuring a heating means affixed within a central vessel of said vaporizer unit where a lower vessel defining a lower portion of said vaporizer unit is insertable into a bowl unit; said bowl unit removably insertable into a stem securely affixed and extending through a chamber top into a chamber, and, a chamber outlet extending through said chamber top. Further disclosed is a vaporizer pipe assembly comprising a vaporizer unit with a central vessel formed from blown glass. Further disclosed is a vaporizer pipe assembly comprising a cage affixed to said vaporizing unit by fastening means. Also disclosed is a vaporizer pipe assembly comprising a central vessel formed from colored glass. Disclosed is a vaporizer pipe assembly wherein said cage is formed from wire, and also a vaporizer pipe assembly wherein said central vessel has at least one air flow aperture formed through a wall of said central vessel. Disclosed is a vaporizer pipe assembly wherein said air flow aperture(s) are in the range of 2 to 10 millimeters in diameter. Disclosed is a vaporizer pipe assembly wherein said cage further comprises a frame member with upper and lower sections with a plurality of cross members affixed therebetween said upper and lower sections and wherein said cage is secured to said vaporizer unit or handle by a fastening means. Disclosed is a vaporizer pipe assembly wherein said bowl unit further comprises an upper opening for receiving said lower vessel, a lower opening at a base of said bowl unit, and a screen receiving means situated therebetween said upper and lower openings. Disclosed is a vaporizer pipe assembly further comprising a hanger. Disclosed is a vaporizer unit comprising: a central vessel with a heating means removably affixed within said central vessel; said heating means electrically connected to a power source; said central vessel featuring at least one air flow aperture disposed thereon; and, a lower vessel defining a lower portion of said vaporizer unit. Also disclosed is a vaporizer unit wherein said central vessel is formed from blown glass, and another embodiment wherein said central vessel is formed from colored glass. Disclosed is a vaporizer unit wherein said central vessel changes colors. Disclosed is a vaporizer further comprising a heating means that is a halogen bulb; and another embodiment where the vaporizer unit further comprises a heating means that may be precisely controlled by use of a dimmer. Disclosed is a vaporizer unit further comprising a handle. Disclosed is a also a vaporizer wherein said handle features a hollow passageway extending therethrough to pass through power cords supplying said heating means with electricity. Disclosed is a vaporizer unit wherein said air flow apertures are covered by a vent covering; and yet another embodiment where said air flow apertures are in the range of 4 to 10 millimeters in diameter. Disclosed further is a cage for a vaporizer unit comprising a frame member fixed to a vaporizer unit by a fastening means and also a another embodiment of the cage further comprising upper and lower sections with a plurality of cross members affixed therebetween.

It is to be noted, however, that the appended drawings illustrate only typical embodiments of this invention, are not to scale, and are therefore not to be considered limiting of its scope, for the invention may admit to other equally effective embodiments which are appreciated by those skillful in the arts.

I claim:

1. A vaporizer pipe assembly comprising:
(a) a vaporizer unit featuring a heating means affixed within a central vessel of said vaporizer unit where a lower vessel defining a lower portion of said vaporizer unit is insertable into a bowl unit;
(b) said bowl unit removably insertable into a stem securely affixed and extending through a chamber top into a chamber, and, (c) a chamber outlet extending through said chamber top.

2. The vaporizer pipe assembly of claim 1 further comprising a vaporizer unit with a central vessel formed from blown glass.

3. The vaporizer pipe assembly of claim 1 further comprising a cage affixed to said vaporizing unit by fastening means.

4. The vaporizer pipe assembly of claim 1 further comprising a central vessel formed from colored glass.

5. The vaporizer pipe assembly of claim 1 wherein said cage is formed from wire.

6. The vaporizer pipe assembly of claim 1 wherein said central vessel has at least one air flow aperture formed through a wall of said central vessel.

7. The vaporizer pipe assembly of claim 1 wherein said air flow aperture(s) are in the range of 2 to 10 millimeters in diameter.

8. The vaporizer pipe assembly of claim 1 wherein said cage further comprises a frame member with upper and lower sections with a plurality of cross members affixed therebetween said upper and lower sections and wherein said cage is secured to said vaporizer unit or handle by a fastening means.

9. The vaporizer pipe assembly of claim 1 wherein said bowl unit further comprises an upper opening for receiving said lower vessel, a lower opening at a base of said bowl unit, and a screen receiving means situated therebetween said upper and lower openings.

10. The vaporizer pipe assembly of claim 1 further comprising a hanger.

11. A vaporizer unit comprising:
(a) a central vessel with a heating means removably affixed within said central vessel;
(b) said heating means electrically connected to a power source;
(c) said central vessel featuring at least one air flow aperture disposed thereon; and,
(d) a lower vessel defining a lower portion of said vaporizer unit.

12. The vaporizer unit of claim 11 wherein said central vessel is formed from blown glass.

13. The vaporizer unit of claim 11 wherein said central vessel is formed from colored glass.

14. The vaporizer unit of claim 11 wherein said central vessel changes colors.

15. The vaporizer unit of claim 11 further comprising a heating means that is a halogen bulb.

16. The vaporizer unit of claim 11 further comprising a heating means that may be precisely controlled by use of a dimmer.

17. The vaporizer unit of claim 11 further comprising a handle.

18. The vaporizer unit of claim 11 wherein said handle features a hollow passageway extending therethrough to pass through power cords supplying said heating means with electricity.

19. The vaporizer unit of claim 11 wherein said air flow apertures are covered by a vent covering.

20. The vaporizer unit of claim 11 wherein said air flow apertures are in the range of 4 to 10 millimeters in diameter.

21. A cage for a vaporizer unit comprising a frame member fixed to a vaporizer unit by a fastening means.

22. The cage of claim 21, further comprising upper and lower sections with a plurality of cross members affixed therebetween.
